(19) United States
(12) Patent Application Publication
(10) Pub. No.: US 2024/0395409 A1
(43) Pub. Date: Nov. 28, 2024

(54) INFORMATION PROCESSING SYSTEM, INFORMATION PROCESSING METHOD, AND INFORMATION PROCESSING PROGRAM

(71) Applicant: FUJIFILM CORPORATION, Tokyo (JP)

(72) Inventor: Hironori MATSUMASA, Tokyo (JP)

(21) Appl. No.: 18/791,450

(22) Filed: Aug. 1, 2024

Related U.S. Application Data
(63) Continuation of application No. PCT/JP2023/004451, filed on Feb. 9, 2023.

(30) Foreign Application Priority Data
Feb. 9, 2022 (JP) 2022-019036

(51) Int. Cl. G16H 50/20; G16H 30/20; G16H 30/40

(52) U.S. Cl. CPC G16H 50/20; G16H 30/40; G16H 30/20

(57) ABSTRACT

An information processing system including: a first information processing apparatus including at least one first processor; and a second information processing apparatus including at least one second processor, wherein: the first processor is configured to perform training of a learning model that receives biological information and outputs diagnosis information by using a combination of biological information including at least a medical image and diagnosis information related to the biological information, and the second processor is configured to: acquire the learning model that is trained from the first information processing apparatus; and generate new diagnosis information related to new biological information that is different from the biological information used for training of the learning model that is trained by inputting the new biological information to the learning model that is trained.

### CROSS REFERENCE TO RELATED APPLICATIONS

This application is a continuation of International Application No. PCT/JP2023/004451, filed on Feb. 9, 2023, which claims priority from Japanese Patent Application No. 2022-019036, filed on Feb. 9, 2022. The entire disclosure of each of the above applications is incorporated herein by reference.

### BACKGROUND

### Technical Field

The present disclosure relates to an information processing system, an information processing method, and an information processing program.

### Related Art

In the related art, image diagnosis is performed using medical images obtained by imaging apparatuses such as computed tomography (CT) apparatuses and magnetic resonance imaging (MRI) apparatuses. In addition, medical images are analyzed via computer aided detection/diagnosis (CAD) using a discriminator obtained by performing training using deep learning or the like, and regions-of-interest including structures, lesions, and the like included in the medical images are detected and/or diagnosed.

In some cases, personal information such as a name, a gender, and an age of a subject is attached to the medical image. JP2021-061042A discloses a system and a method for anonymizing health data in order to protect privacy of a patient in a case of transferring the health data from one geographical region to another geographical region for data analysis.

In the related art, research and development of a learning model (discriminator) for CAD have been performed at research institutions of manufacturers or the like, and operation of the learning model has been performed at medical institutions such as a hospital. In order to research and develop the learning model, it is necessary to collect training data. However, since biological information to be used as the training data is collected in a medical institution, there is a need for cooperation between the research institution and the medical institution. In addition, it is easy to find an error in an output result of the learning model during the operation at the medical institution. However, in order to correct the error and improve accuracy of the learning model, it is necessary to perform retraining of the learning model in the research institution. As a result, this leads to the need for cooperation between the research institution and the medical institution.

### SUMMARY

The present disclosure provides an information processing system, an information processing method, and an information processing program that can consistently manage training and operation of a learning model.

According to a first aspect of the present disclosure, there is provided an information processing system including: a first information processing apparatus including at least one first processor; and a second information processing apparatus including at least one second processor, in which the first processor is configured to: perform training of a learning model that receives biological information and outputs diagnosis information by using a combination of biological information including at least a medical image and diagnosis information related to the biological information, and the second processor is configured to: acquire the learning model that is trained from the first information processing apparatus, and generate new diagnosis information related to new biological information that is different from the biological information used for training of the learning model that is trained by inputting the new biological information to the learning model that is trained.

According to a second aspect of the present disclosure, in the first aspect, the first information processing apparatus may further include an input unit, the second information processing apparatus may further include a storage unit that stores the biological information, and the first processor may be configured to: acquire the biological information stored in the storage unit from the second information processing apparatus, display the biological information on a display, and receive, via the input unit, an input for diagnosis information related to the displayed biological information.

According to a third aspect of the present disclosure, in the first aspect or the second aspect, the first processor may be configured to: acquire a combination of the new biological information and the new diagnosis information, and perform retraining of the learning model by using the combination of the new biological information and the new diagnosis information.

According to a fourth aspect of the present disclosure, in any one of the first aspect to the third aspect, the second information processing apparatus may further include an input unit, and the second processor may be configured to: display the generated new diagnosis information on a display, and receive, via the input unit, a correction for the new diagnosis information.

According to a fifth aspect of the present disclosure, in the fourth aspect, the first processor may be configured to: acquire a combination of the new biological information and the new diagnosis information, and perform, in a case where the new diagnosis information is corrected, retraining of the learning model by using a combination of the new biological information and the corrected new diagnosis information.

According to a sixth aspect of the present disclosure, in any one of the first aspect to the fifth aspect, accessory information may be attached to the biological information, the accessory information indicating information related to at least one of a subject from which the biological information is acquired or an imaging apparatus used for acquisition of the biological information.

According to a seventh aspect of the present disclosure, in the sixth aspect, the information processing system may further include: a third information processing apparatus including at least one third processor. The third processor may be configured to anonymize at least a part of the accessory information attached to the biological information.

According to an eighth aspect of the present disclosure, in the seventh aspect, the second information processing apparatus may further include a storage unit that stores the biological information and the accessory information, the third processor may be configured to: acquire the accessory information from the second information processing apparatus, and anonymize at least a part of the accessory information, the second processor may be configured to: acquire the accessory information after anonymization from the third information processing apparatus, and store, in the storage unit, the biological information, the accessory information before anonymization, and the accessory information after anonymization in association with each other, and the first processor may be configured to: acquire the biological information to which the accessory information after anonymization is attached.

According to a ninth aspect of the present disclosure, in the seventh aspect or the eighth aspect, the third processor may be configured to: acquire accessory information attached to the new biological information from the second information processing apparatus, and anonymize at least a part of the accessory information, and the first processor may be configured to: acquire the new biological information to which the accessory information after anonymization is attached, and perform retraining of the learning model by using a combination of the new biological information and the new diagnosis information.

According to a tenth aspect of the present disclosure, in any one of the sixth aspect to the ninth aspect, the second processor may be configured to: estimate a similarity between the accessory information attached to the biological information used for training of the learning model and the accessory information attached to the new biological information.

According to an eleventh aspect of the present disclosure, in the tenth aspect, the second processor may be configured to present the generated new diagnosis information and the estimated similarity.

According to a twelfth aspect of the present disclosure, in the tenth aspect or the eleventh aspect, the first processor may be configured to: acquire a combination of the new biological information, the new diagnosis information, and the similarity, and determine whether or not to perform retraining of the learning model by using the combination of the new biological information and the new diagnosis information based on the similarity.

According to a thirteenth aspect of the present disclosure, in any one of the sixth aspect to the twelfth aspect, the accessory information may include information indicating at least one of a name, a gender, an age, a medical history, and an identification number of the subject, or an imaging condition used for acquiring the biological information.

According to a fourteenth aspect of the present disclosure, in any one of the first aspect to the thirteenth aspect, the biological information may include pre-diagnosis information indicating information obtained by performing diagnosis in advance in relation to the medical image included in the biological information, and the first processor may be configured to: perform training of the learning model that receives the biological information and outputs the diagnosis information by using a combination of the medical image included in the biological information, the pre-diagnosis information, and the diagnosis information related to the biological information.

According to a fifteenth aspect of the present disclosure, in any one of the first aspect to the fourteenth aspect, the diagnosis information may include at least one of information indicating a position and a size of a region-of-interest included in the medical image or information indicating an opinion of the region-of-interest.

According to a sixteenth aspect of the present disclosure, there is provided an information processing method including: causing a first processor to perform training of a learning model that receives biological information and outputs diagnosis information by using a combination of biological information including at least a medical image and diagnosis information related to the biological information; and causing a second processor to acquire the learning model that is trained by the first processor and generate new diagnosis information related to new biological information, which is different from the biological information used for training of the learning model that is trained, by inputting the new biological information to the learning model that is trained.

According to a seventeenth aspect of the present disclosure, there is provided an information processing program including: causing a first processor to execute processing of performing training of a learning model that receives biological information and outputs diagnosis information by using a combination of biological information including at least a medical image and diagnosis information related to the biological information; and causing a second processor to execute processing of acquiring the learning model that is trained by the first processor and generating new diagnosis information related to new biological information, which is different from the biological information used for training of the learning model that is trained, by inputting the new biological information to the learning model that is trained.

According to the above aspects, the information processing system, the information processing method, and the information processing program of the present disclosure can consistently manage training and operation of the learning model.

### BRIEF DESCRIPTION OF THE DRAWINGS

FIG. 1 is a diagram illustrating an example of a schematic configuration of an information processing system according to first and second exemplary embodiments.

FIG. 2 is a block diagram illustrating an example of a hardware configuration of a research server.

FIG. 3 is a block diagram illustrating an example of a hardware configuration of a clinical server.

FIG. 4 is a block diagram illustrating an example of a functional configuration of the research server and the clinical server in a training phase.

FIG. 5 is a diagram illustrating an example of biological information.

FIG. 6 is a diagram illustrating an example of a screen displayed on a display.

FIG. 7 is a block diagram illustrating an example of a functional configuration of the research server and the clinical server in an operation phase.

FIG. 8 is a diagram illustrating an example of a screen displayed on a display.

FIG. 9 is a diagram illustrating an example of a screen displayed on a display.

FIG. 10 is a block diagram illustrating an example of a functional configuration of the research server and the clinical server in a retraining phase.

FIG. 11 is a flowchart illustrating an example of 1-1 information processing.

FIG. 12 is a flowchart illustrating an example of 1-2 information processing.

FIG. 13 is a flowchart illustrating an example of 2-1 information processing.

FIG. 14 is a flowchart illustrating an example of 2-2 information processing.

FIG. 15 is a flowchart illustrating an example of 3-1 information processing.

FIG. 16 is a flowchart illustrating an example of 3-2 information processing.

FIG. 17 is a block diagram illustrating an example of a functional configuration of the research server and the clinical server according to the second exemplary embodiment.

FIG. 18 is a diagram illustrating an example of a similarity between accessory information.

FIG. 19 is a diagram illustrating an example of a screen displayed on a display.

FIG. 20 is a diagram illustrating an example of a schematic configuration of the information processing system according to a third exemplary embodiment.

FIG. 21 is a block diagram illustrating an example of a hardware configuration of an anonymization server.

FIG. 22 is a block diagram illustrating an example of a functional configuration of the research server, the clinical server, and the anonymization server.

FIG. 23 is a diagram illustrating an example of accessory information after anonymization.

### DETAILED DESCRIPTION

Hereinafter, each of exemplary embodiments of the present disclosure will be described with reference to the drawings.

### First Exemplary Embodiment

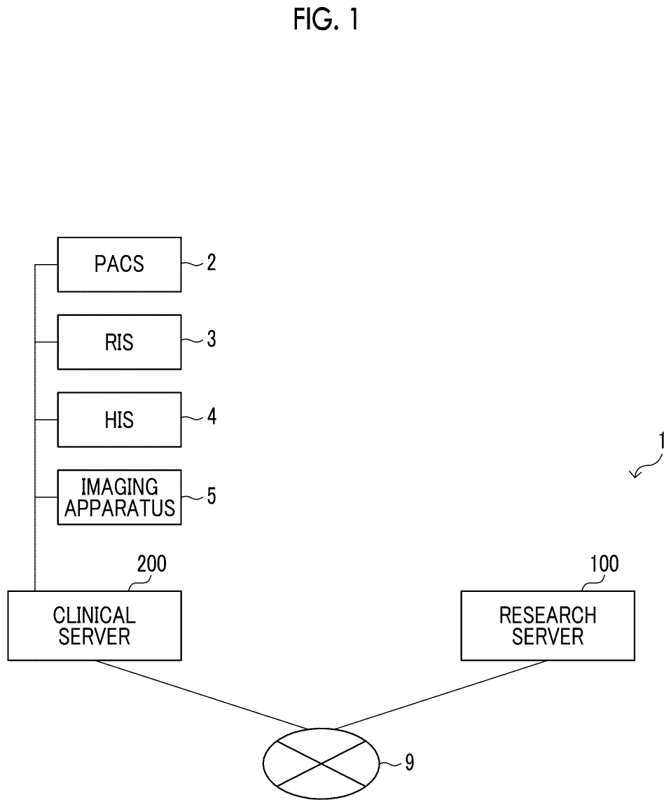

First, a configuration of an information processing system 1 according to the present disclosure will be described. FIG. 1 is a diagram illustrating a schematic configuration of the information processing system 1. As illustrated in FIG. 1, the information processing system 1 includes a research server 100 and a clinical server 200. The research server 100 and the clinical server 200 are connected to each other via a wired or wireless network 9 such that communication can be performed. The network 9 is, for example, a network such as a local area network (LAN) and a wide area network (WAN). The research server 100 is an example of a first information processing apparatus according to the present disclosure, and the clinical server 200 is an example of a second information processing apparatus according to the present disclosure.

The clinical server 200 is connected to a known medical information system, such as a picture archiving and communication system (PACS) 2, a radiology information system (RIS) 3, and a hospital information system (HIS) 4. In addition, the clinical server 200 may be connected to an imaging apparatus 5 (modality) that generates a medical image representing a diagnosis target site by imaging a site that is a diagnosis target of a subject. The imaging apparatus 5 is, for example, a simple X-ray imaging apparatus, a computed tomography (CT) apparatus, a magnetic resonance imaging (MRI) apparatus, a positron emission tomography (PET) apparatus, or the like.

Here, various external apparatuses, such as the PACS 2, the RIS 3, the HIS 4, and the imaging apparatus 5, are apparatuses that are actually in operation in a medical institution or the like (so-called an operating environment). On the other hand, the research server 100 is not connected to the operating environment. The information processing system 1 according to the present disclosure is used for training of a diagnosis model M that performs diagnosis related to biological information by using the biological information obtained in the operating environment.

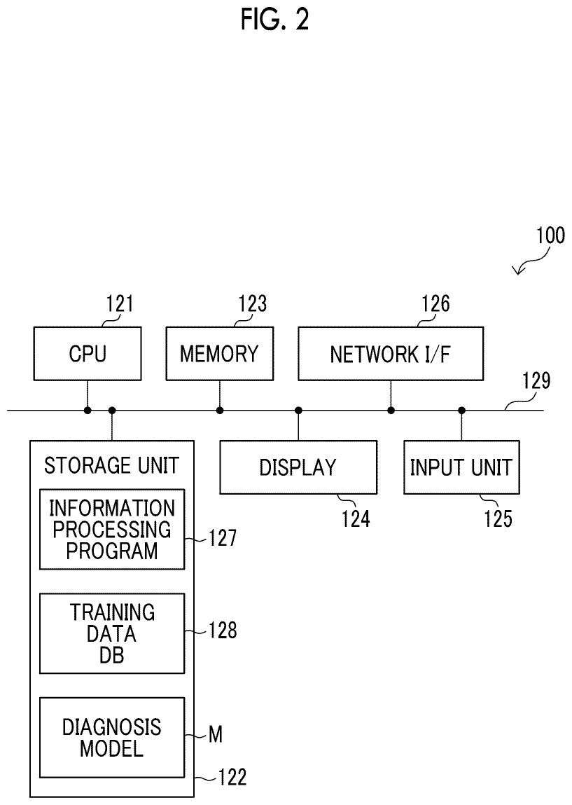

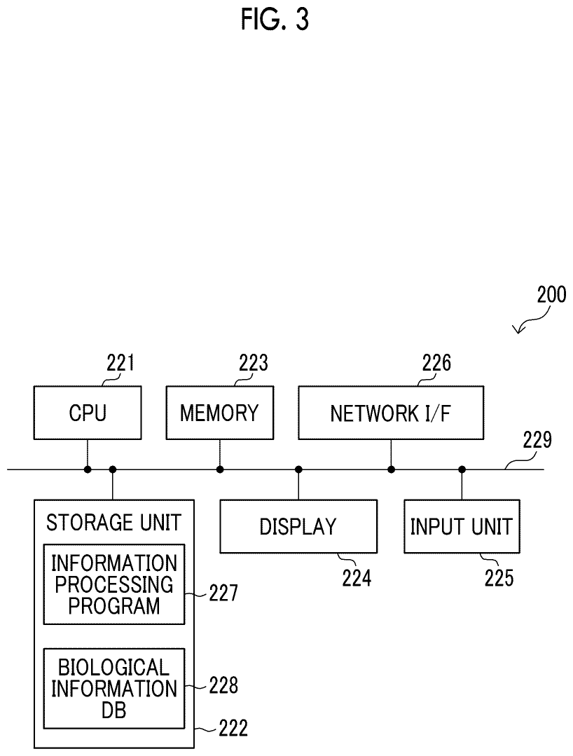

Next, an example of a hardware configuration of the research server 100 and the clinical server 200 will be described with reference to FIG. 2 and FIG. 3. As the research server 100 and the clinical server 200, for example, a server computer, a personal computer, and the like can be appropriately applied.

As illustrated in FIG. 2, the research server 100 includes a central processing unit (CPU) 121, a non-volatile storage unit 122, and a memory 123 as a temporary storage region. In addition, the research server 100 includes a display 124 such as a liquid crystal display, an input unit 125 such as a keyboard and a mouse, and a network interface (I/F) 126. The network I/F 126 is connected to the network 9, and performs wired or wireless communication. The CPU 121, the storage unit 122, the memory 123, the display 124, the input unit 125, and the network I/F 126 are connected to each other via a bus 129 such as a system bus and a control bus such that various types of information can be exchanged.

The storage unit 122 is realized by, for example, a storage medium, such as a hard disk drive (HDD), a solid state drive (SSD), and a flash memory. The storage unit 122 stores an information processing program 127 in the research server 100. The CPU 121 reads out the information processing program 127 from the storage unit 122, develops the read information processing program 127 in the memory 123, and executes the developed information processing program 127. The CPU 121 is an example of a first processor according to the present disclosure.

In addition, the storage unit 122 stores a training data database (DB) 128 and a diagnosis model M. The training data DB 128 stores training data to be used for training of the diagnosis model M (details will be described below). Note that the storage unit 122 may be realized by, for example, a cloud server.

The diagnosis model M is a model that receives the biological information and outputs information (hereinafter, referred to as “diagnosis information”) related to diagnosis of the biological information. The diagnosis model M is configured with, for example, a neural network such as a convolutional neural network (CNN) and a recurrent neural network (RNN). The diagnosis model M is an example of a learning model according to the present disclosure.

As illustrated in FIG. 3, the clinical server 200 includes a CPU 221, a non-volatile storage unit 222, and a memory 223 as a temporary storage region. In addition, the clinical server 200 includes a display 224 such as a liquid crystal display, an input unit 225 such as a keyboard and a mouse, and a network I/F 226. The network I/F 226 is connected to the network 9, and performs wired or wireless communication. The CPU 221, the storage unit 222, the memory 223, the display 224, the input unit 225, and the network I/F 226 are connected to each other via a bus 229 such as a system bus and a control bus such that various types of information can be exchanged.

The storage unit 222 is realized by, for example, a storage medium, such as an HDD, an SSD, and a flash memory. The storage unit 222 stores an information processing program 227 in the clinical server 200. The CPU 221 reads out the information processing program 227 from the storage unit 222, develops the read information processing program 227 in the memory 223, and executes the developed information processing program 227. The CPU 221 is an example of a second processor according to the present disclosure.

In addition, the storage unit 222 stores a biological information DB 228. The biological information DB 228 stores the biological information acquired from various external apparatuses such as the PACS 2, the RIS 3, the HIS 4, and the imaging apparatus 5 (details will be described below). That is, the storage unit 222 stores the biological information. Note that the storage unit 222 may be realized by, for example, a cloud server.

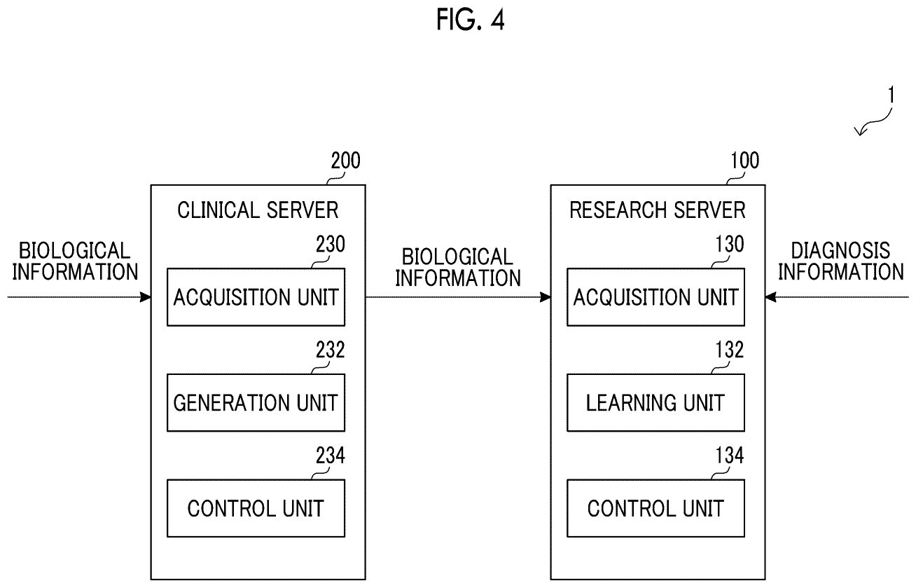

Next, an example of a functional configuration of the research server 100 and the clinical server 200 according to the present exemplary embodiment will be described with reference to FIG. 4 to FIG. 10. As illustrated in FIG. 4, the research server 100 includes an acquisition unit 130, a learning unit 132, and a control unit 134. In a case where the CPU 121 executes the information processing program 127, the CPU 121 functions as the acquisition unit 130, the learning unit 132, and the control unit 134. In addition, as illustrated in FIG. 4, the clinical server 200 includes an acquisition unit 230, a generation unit 232, and a control unit 234. In a case where the CPU 221 executes the information processing program 227, the CPU 221 functions as the acquisition unit 230, the generation unit 232, and the control unit 234. In the following description, functions of the research server 100 and the clinical server 200 will be described in a training phase, an operation phase, and a retraining phase of the diagnosis model M.

### (Training Phase)

First, functions of the research server 100 and the clinical server 200 in the training phase of the diagnosis model M will be described with reference to FIG. 4 to FIG. 6. FIG. 4 also illustrates various types of information to be input and output to and from the research server 100 and the clinical server 200 in the training phase. In the training phase, in the research server 100, a correct answer label is input and training of the diagnosis model M is performed by using the biological information acquired in the clinical server 200.

The acquisition unit 230 of the clinical server 200 acquires the biological information from various external apparatuses such as the PACS 2, the RIS 3, the HIS 4, and the imaging apparatus 5. The control unit 234 of the clinical server 200 stores the biological information acquired by the acquisition unit 230 in the biological information DB 228 of the storage unit 222. In addition, the control unit 234 outputs the biological information stored in the biological information DB 228 to the research server 100.

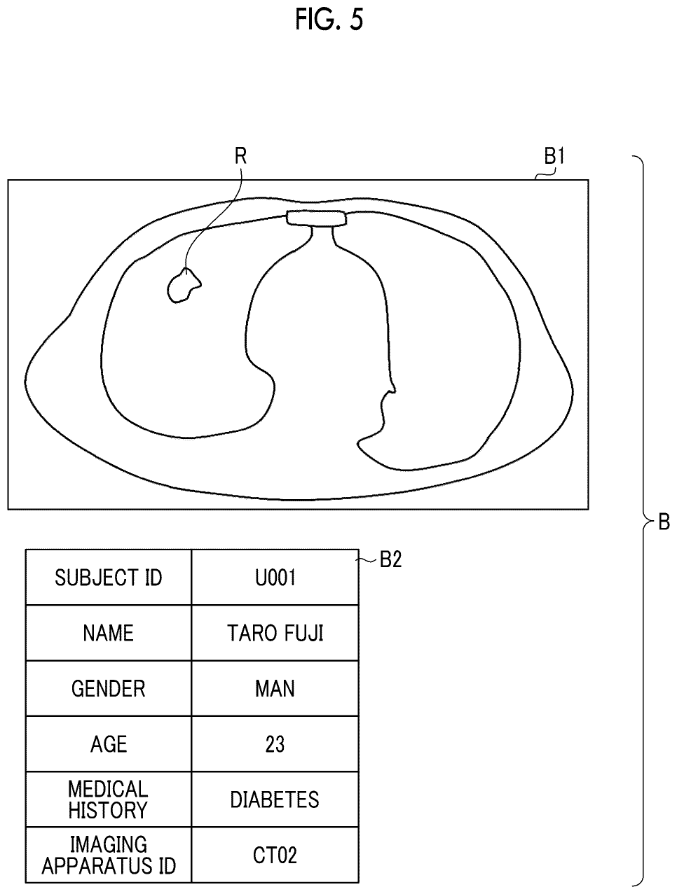

FIG. 5 illustrates an example of the biological information. As illustrated in FIG. 5, the biological information B includes at least a medical image B1. The medical image B1 illustrated in FIG. 5 is a diagram schematically illustrating an example of the medical image acquired by the imaging apparatus 5, and represents a tomographic plane including a lung. The medical image may include various organs (for example, organs such as a lung and a liver) of a human body, and a region of a structure that indicates various tissues and the like (for example, a blood vessel, a nerve, a muscle, and the like) included in the various organs. In addition, the medical image can include a region of an abnormal shadow caused by a lesion (for example, a nodule, a tumor, a damage, a defect, an inflammation, or the like) or the like. In the following description, at least one of the region of the structure or the region of the abnormal shadow is referred to as a “region-of-interest”. In the example of FIG. 5, a region of an abnormal shadow due to a nodule in the medical image B1 is illustrated as a region-of-interest R. Note that one medical image may include a plurality of regions-of-interest.

In addition, as illustrated in FIG. 5, accessory information B2 is attached to the biological information B, the accessory information B2 indicating information related to at least one of a subject from which the biological information is acquired or the imaging apparatus 5 used for the acquisition. For example, the accessory information B2 may include information indicating at least one of a name, a gender, an age, a medical history, and an identification number (“subject identification (ID)”) of the subject, or an imaging condition used for acquiring the biological information. The imaging condition includes, for example, an imaging date and time, a type, a manufacturer, and an identification number (“imaging apparatus ID”) of the imaging apparatus 5 used for capturing the medical image, an imaging site, an imaging protocol, an imaging sequence, an imaging method, the presence or absence of a contrast medium, and the like.

The acquisition unit 130 of the research server 100 acquires the biological information stored in the storage unit 222 (the biological information DB 228) from the clinical server 200. The control unit 134 of the research server 100 performs control of displaying the biological information acquired by the acquisition unit 130 on the display 124. In addition, the control unit 134 receives, as an input, the diagnosis information (that is, the correct answer label) related to the biological information displayed on the display 124, via the input unit 125. The diagnosis information includes, for example, at least one of coordinate information indicating a position and a size of the region-of-interest included in the medical image included in the biological information, or opinion information indicating an opinion of the region-of-interest.

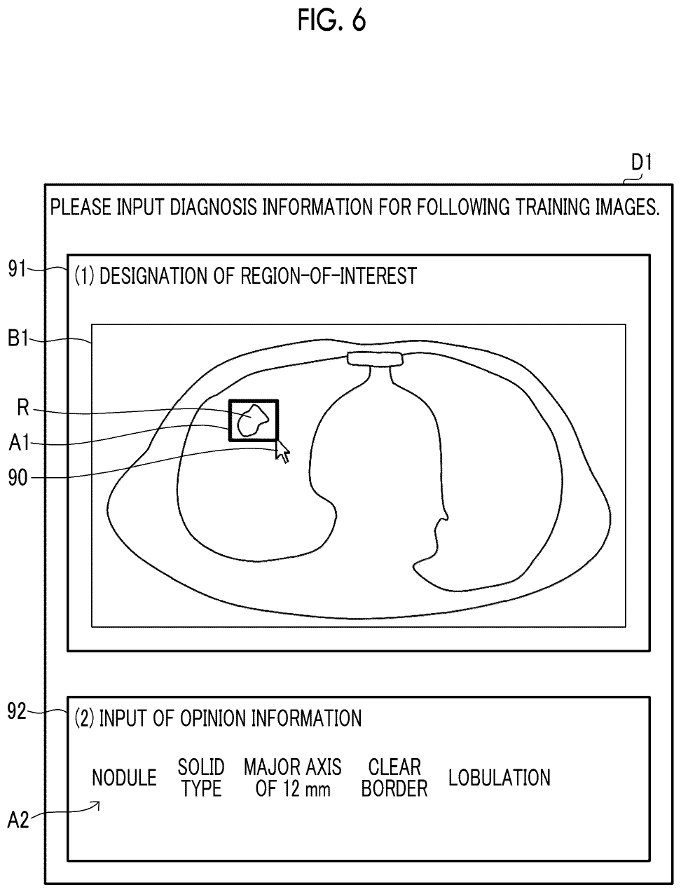

FIG. 6 illustrates an example of a screen D1 displayed on the display 124 by the control unit 134. The screen D1 includes the medical image B1 including the region-of-interest R and input fields 91 and 92 for diagnosis information related to the medical image B1. The input field 91 is a field for inputting, as an example of the diagnosis information, coordinate information A1 indicating a position and a size of the region-of-interest R included in the medical image B1. Specifically, the control unit 134 may receive designation of the coordinate information A1 in a form, such as a bounding box and a mask, in response to an operation of a mouse pointer 90 in the input field 91.

The input field 92 is a field for inputting, as an example of the diagnosis information, opinion information A2 related to the medical image B1 and/or the region-of-interest R. The opinion information A2 may include, for example, information indicating at least one of a name (type), a property, a measured value, a position, or an estimated disease name of the medical image B1 and/or the region-of-interest R. Examples of the name (type) include a name of a structure such as “lung” and “liver”, and a name of an abnormal shadow such as “nodule”. The property mainly means a feature of an abnormal shadow. For example, in a case of a lung nodule, opinions indicating absorption values such as “solid type” and “ground-glass type”, “clear/unclear”, “smooth/irregular”, “spicula”, a margin shape such as “lobulation” and “serration”, and an overall shape such as “almost circular shape” and “irregular shape” can be mentioned. In addition, for example, there are opinions related to a relationship with surrounding tissues, such as “pleural contact” and “pleural invagination”, and the presence or absence of a contrast medium, washout, and the like.

The measured value is a value that can be quantitatively measured from a medical image, and examples of the measured value include a size (a major axis, a minor axis, a volume, and the like), a CT value of which a unit is HU, the number of regions-of-interest in a case where there are a plurality of regions-of-interest, and a distance between regions-of-interest. Further, the measured value may be replaced with a qualitative expression such as “large/small” or “more/less”. The position means an anatomical position, a position in a medical image, and a relative positional relationship with other regions-of-interest, such as “inside”, “margin”, and “periphery”. The anatomical position may be indicated by an organ name such as “lung” and “liver”, and may be expressed in terms of subdivided names such as “right lung”, “upper lobe”, and apical segment (“S1”). The estimated disease name is an evaluation result estimated based on the abnormal shadow. Examples of the estimated disease name include a disease name such as “cancer” and “inflammation” and an evaluation result such as “negative/positive”, “benign/malignant”, and “mild/severe” related to disease names and properties.

The learning unit 132 of the research server 100 performs training of the diagnosis model M by using a combination of the biological information that is acquired by the acquisition unit 130 and the diagnosis information that is received by the control unit 134 and is related to the biological information. The control unit 134 stores the combination of the biological information to be used for the training of the diagnosis model M and the diagnosis information in the training data DB 128 of the storage unit 122.

### (Operation Phase)

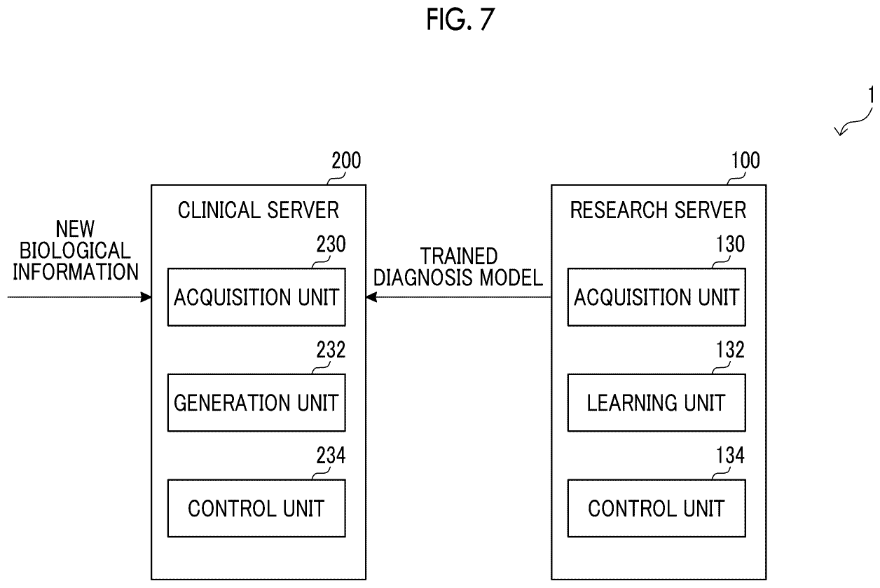

Next, functions of the research server 100 and the clinical server 200 in the operation phase of the diagnosis model M will be described with reference to FIG. 7 to FIG. 9. FIG. 7 also illustrates various types of information to be input and output to and from the research server 100 and the clinical server 200 in the operation phase. In the operation phase, diagnosis of the biological information which is newly obtained in the operating environment is performed by using the diagnosis model M obtained by performing training in the research server 100.

The control unit 134 of the research server 100 outputs the diagnosis model M obtained by performing training in the training phase, to the clinical server 200. The acquisition unit 230 of the clinical server 200 acquires the trained diagnosis model M from the research server 100.

In addition, the acquisition unit 230 acquires new biological information that is different from the biological information used for training of the diagnosis model M on which training is performed in the training phase, from various external apparatuses such as the PACS 2, the RIS 3, the HIS 4, and the imaging apparatus 5. The generation unit 232 of the clinical server 200 generates new diagnosis information related to the new biological information by inputting the new biological information acquired by the acquisition unit 230 to the trained diagnosis model M that is acquired by the acquisition unit 230.

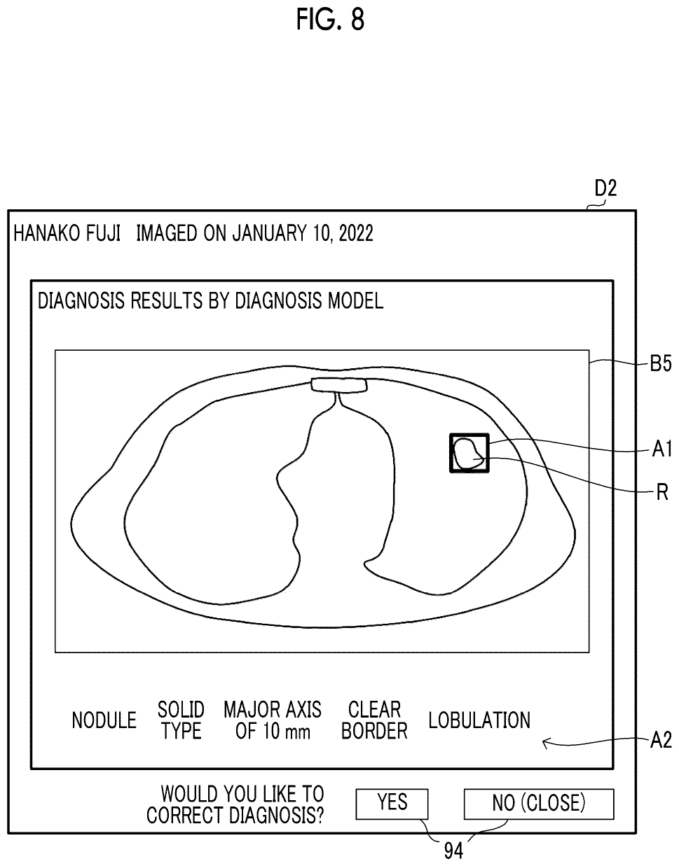

The control unit 234 of the clinical server 200 performs control of displaying the new diagnosis information generated by the generation unit 232 on the display 224. FIG. 8 illustrates an example of a screen D2 displayed on the display 224 by the control unit 234. The coordinate information A1 of the region-of-interest R that is an example of the new diagnosis information is displayed on a medical image B5 (new biological information) on the screen D2. In addition, the opinion information A2 of the medical image B5 and/or the region-of-interest R that is an example of the new diagnosis information is displayed on the screen D2. In addition, the control unit 234 stores the new biological information and the new diagnosis information in the biological information DB 228 of the storage unit 222 by associating pieces of the information.

In addition, the control unit 234 may receive correction of the new diagnosis information displayed on the display 224 by the user via the input unit 225. A button 94 for selecting whether or not to receive the correction is displayed on the screen D2. In a case where the user designates to receive the correction of the new diagnosis information via the button 94, the control unit 234 performs control of displaying a screen for receiving the correction of the new diagnosis information on the display 224.

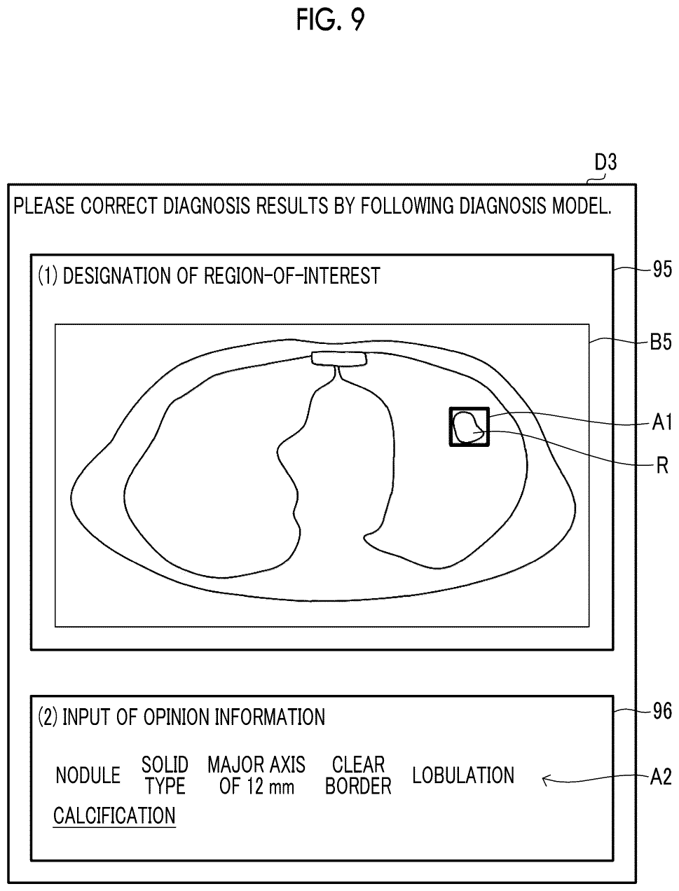

FIG. 9 illustrates an example of a screen D3 displayed on the display 224 by the control unit 234. The screen D3 includes an input field 95 for receiving correction of the coordinate information A1 and an input field 96 for receiving correction of the opinion information A2. In a case where correction of the coordinate information A1 and/or the opinion information A2 is received, the control unit 234 stores the corrected new diagnosis information and the new biological information in the biological information DB 228 of the storage unit 222 by associating pieces of the information.

### (Retraining Phase)

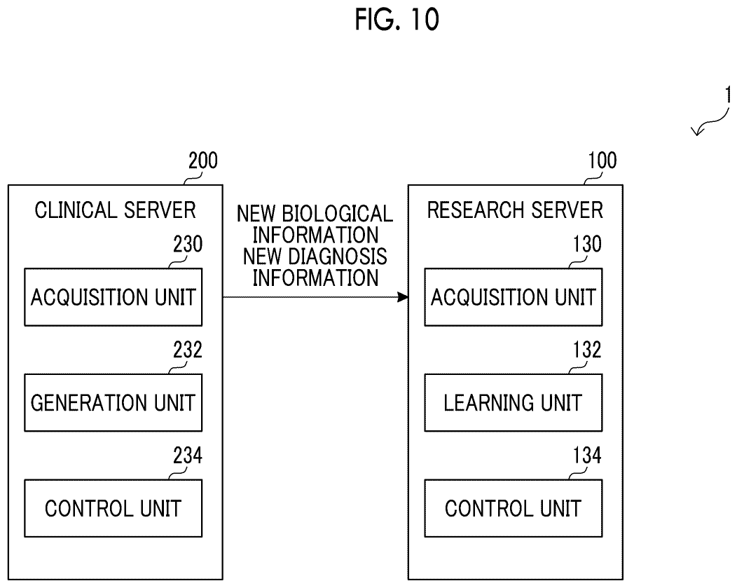

Next, functions of the research server 100 and the clinical server 200 in a retraining phase of the diagnosis model M will be described with reference to FIG. 10. FIG. 10 also illustrates various types of information to be input and output to and from the research server 100 and the clinical server 200 in a retraining phase. In the retraining phase, retraining of the trained diagnosis model M is performed in the research server 100 by using a combination of the new biological information and the new diagnosis information.

The control unit 234 of the clinical server 200 outputs the new diagnosis information and the new biological information stored in the biological information DB 228 to the research server 100. The acquisition unit 130 of the research server 100 acquires the combination of the new biological information and the new diagnosis information from the clinical server 200. The learning unit 132 of the research server 100 performs retraining of the diagnosis model M by using the combination of the new biological information and the new diagnosis information that is acquired by the acquisition unit 130.

Note that, as described above, the new diagnosis information may be corrected by the user in the clinical server 200. In a case where the new diagnosis information is corrected, the learning unit 132 of the research server 100 may perform retraining of the diagnosis model M by using the combination of the new biological information and the corrected new diagnosis information.

In a case where retraining of the diagnosis model M is performed and then the diagnosis model M is operated again in the clinical server 200, the control unit 134 of the research server 100 outputs the retrained diagnosis model M obtained by performing retraining in the retraining phase, to the clinical server 200. The clinical server 200 generates new diagnosis information by using the retrained diagnosis model M that is acquired from the research server 100. In this way, retraining of the diagnosis model M is performed by using the combination of the new biological information and the new diagnosis information that is generated in the operation phase, and the retrained diagnosis model M is operated. This processing is repeatedly performed, and thus accuracy of the diagnosis model M can be improved.

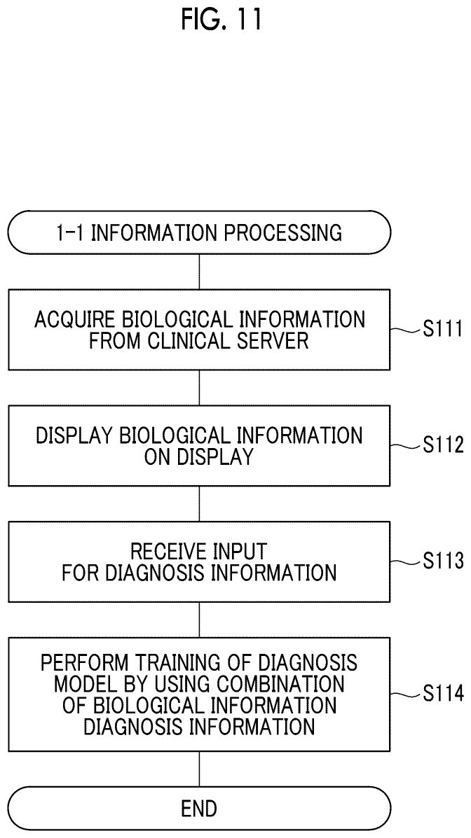

Next, actions of the research server 100 and the clinical server 200 according to the present exemplary embodiment will be described with reference to FIG. 11 to FIG. 16. In the following description, actions of the research server 100 and the clinical server 200 will be described in a training phase, an operation phase, and a retraining phase of the diagnosis model M.

### (Training Phase)

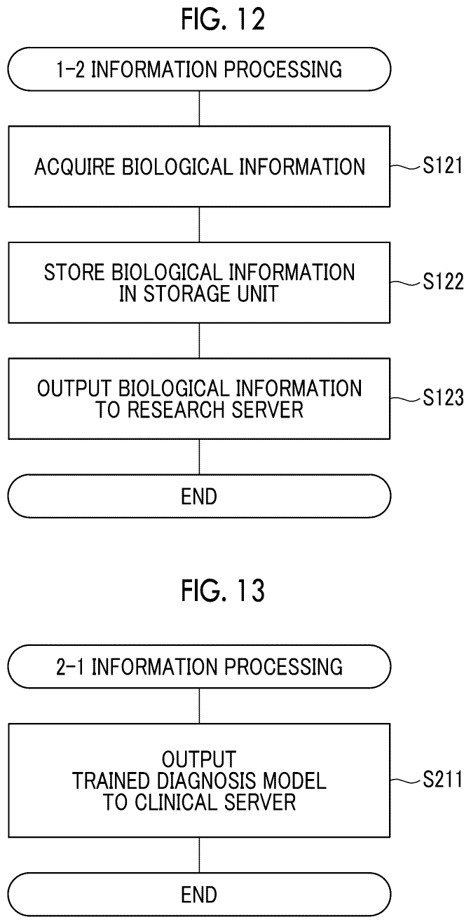

In the research server 100, the CPU 121 executes the information processing program 127, and thus 1-1 information processing illustrated in FIG. 11 is executed. In the clinical server 200, the CPU 221 executes the information processing program 227, and thus 1-2 information processing illustrated in FIG. 12 is executed. The 1-2 information processing is executed, for example, in a case where the user inputs an instruction to start the execution via the input unit 225, and the 1-1 information processing is executed after the 1-2 information processing.

The 1-2 information processing to be executed by the clinical server 200 will be described with reference to FIG. 12. In step S121, the acquisition unit 230 acquires the biological information from various external apparatuses such as the PACS 2, the RIS 3, the HIS 4, and the imaging apparatus 5. In step S122, the control unit 234 stores the biological information acquired in step S121 in the storage unit 222. In step S123, the control unit 234 outputs the biological information stored in the storage unit 222 in step S121 to the research server 100, and ends the 1-2 information processing.

The 1-1 information processing to be executed by the research server 100 will be described with reference to FIG. 11. In step S111, the acquisition unit 130 acquires the biological information from the clinical server 200. In step S112, the control unit 134 performs control of displaying the biological information acquired in step S111 on the display 124. In step S113, the control unit 134 receives an input for the diagnosis information related to the biological information displayed on the display 124 in step S112. In step S114, the learning unit 132 performs training of the diagnosis model M by using the combination of the biological information acquired in step S111 and the diagnosis information received in step S113, and ends the 1-1 information processing.

### (Operation Phase)

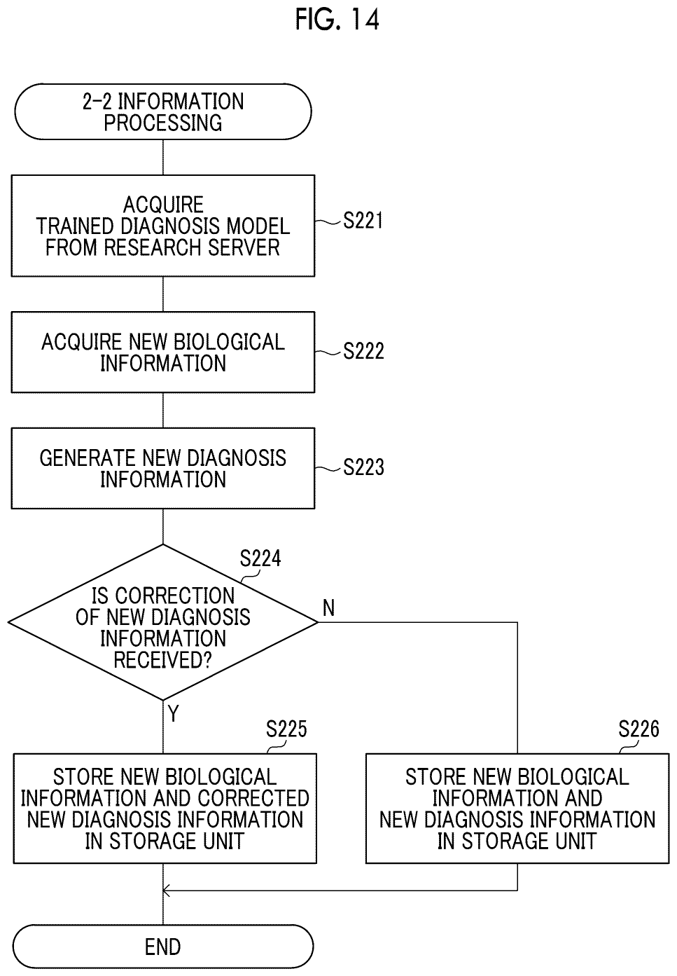

In the research server 100, the CPU 121 executes the information processing program 127, and thus 2-1 information processing illustrated in FIG. 13 is executed. In the clinical server 200, the CPU 221 executes the information processing program 227, and thus 2-2 information processing illustrated in FIG. 14 is executed. The 2-1 information processing is executed, for example, in a case where the user inputs an instruction to start the execution via the input unit 125, and the 2-2 information processing is executed after the 2-1 information processing.

The 2-1 information processing to be executed by the research server 100 will be described with reference to FIG. 13. In step S211, the control unit 134 outputs the diagnosis model M that is trained in the 1-1 information processing to the clinical server 200, and ends the present 2-1 information processing.

The 2-2 information processing to be executed by the clinical server 200 will be described with reference to FIG. 14. In step S221, the acquisition unit 230 acquires the trained diagnosis model M from the research server 100. In step S222, the acquisition unit 230 acquires new biological information from various external apparatuses such as the PACS 2, the RIS 3, the HIS 4, and the imaging apparatus 5. In step S223, the generation unit 232 generates new diagnosis information related to the new biological information by inputting the new biological information acquired in step S222 to the trained diagnosis model M acquired in step S221.

In step S224, the control unit 234 performs control of displaying the new diagnosis information generated in step S223 on the display 224, and receives a correction of the new diagnosis information by the user. In a case where a correction of the new diagnosis information is received (that is, in a case where a determination result in step S224 is Y), the processing proceeds to step S225, and the new biological information acquired in step S222 and the corrected new diagnosis information received in step S224 are stored in the storage unit 222 by being associated with each other.

On the other hand, in a case where a correction of the new diagnosis information is not received (that is, in a case where a determination result in step S224 is N), the processing proceeds to step S226, and the new biological information acquired in step S222 and the new diagnosis information generated in step S223 are stored in the storage unit 222 by being associated with each other. In a case where step S225 and step S226 are completed, the 2-2 information processing is ended.

### (Retraining Phase)

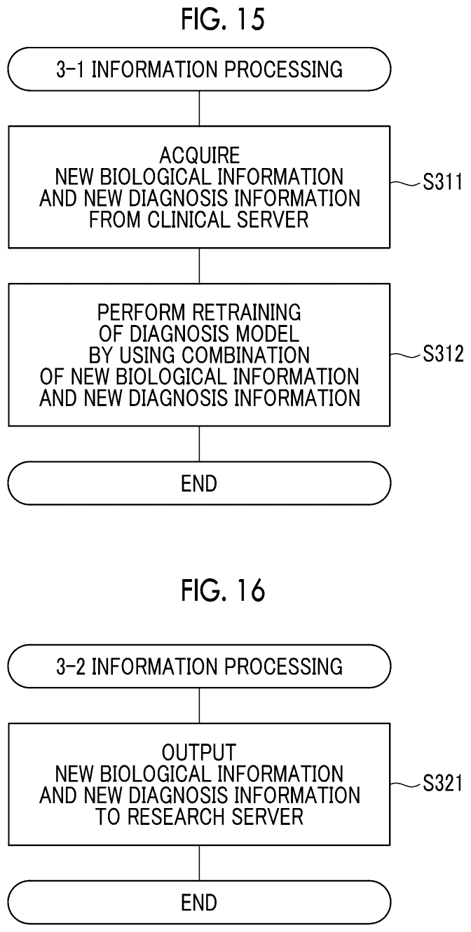

In the research server 100, the CPU 121 executes the information processing program 127, and thus 3-1 information processing illustrated in FIG. 15 is executed. In the clinical server 200, the CPU 221 executes the information processing program 227, and thus 3-2 information processing illustrated in FIG. 16 is executed. The 3-2 information processing is executed, for example, in a case where the user inputs an instruction to start the execution via the input unit 225, and the 3-1 information processing is executed after the 3-2 information processing.

The 3-2 information processing to be executed by the clinical server 200 will be described with reference to FIG. 16. In step S321, the control unit 234 outputs the new diagnosis information and the new biological information stored in the storage unit 222 in the 2-2 information processing to the research server 100, and ends the present 3-2 information processing.

The 3-1 information processing to be executed by the research server 100 will be described with reference to FIG. 15. In step S311, the acquisition unit 130 acquires the combination of the new biological information and the new diagnosis information from the clinical server 200. In step S312, the learning unit 132 performs retraining of the diagnosis model M by using the combination of the new biological information and the new diagnosis information that is acquired in step S311, and ends the 3-1 information processing.

As described above, the information processing system 1 according to an aspect of the present disclosure includes the first information processing apparatus including at least one first processor, and the second information processing apparatus including at least one second processor. The first processor performs training of the learning model that receives the biological information and outputs the diagnosis information by using a combination of the biological information including at least a medical image and the diagnosis information related to the biological information. The second processor acquires the learning model that is trained from the first information processing apparatus, and generates new diagnosis information related to new biological information by inputting, to the learning model that is trained, the new biological information which is different from the biological information used for the training of the learning model that is trained. With such an information processing system 1, the training and the operation of the learning model can be consistently managed.

For example, by utilizing the biological information collected by the clinical server 200, the training of the diagnosis model M in the research server 100 is easily performed. In addition, for example, in a case where an error in the new diagnosis information is discovered in the operation of the diagnosis model M in the clinical server 200, the research server 100 performs retraining of the diagnosis model M based on the new diagnosis information in which the error is corrected. Thus, the accuracy of the diagnosis model M can be improved.

Note that, in the first exemplary embodiment, the accessory information may not be attached to the biological information (the medical image).

### Second Exemplary Embodiment

The information processing system 1 according to the present exemplary embodiment has a function of estimating a similarity between pieces of accessory information that are attached to each of the biological information used for the training of the diagnosis model M and the new biological information for which the new diagnosis information is generated in the operation phase. This is because the similarity between the pieces of accessory information is considered to be correlated with accuracy of the new diagnosis information. In the following description, the information processing system 1 according to the second exemplary embodiment will be described. On the other hand, descriptions of the same configurations and functions as those of the first exemplary embodiment will be omitted as appropriate.

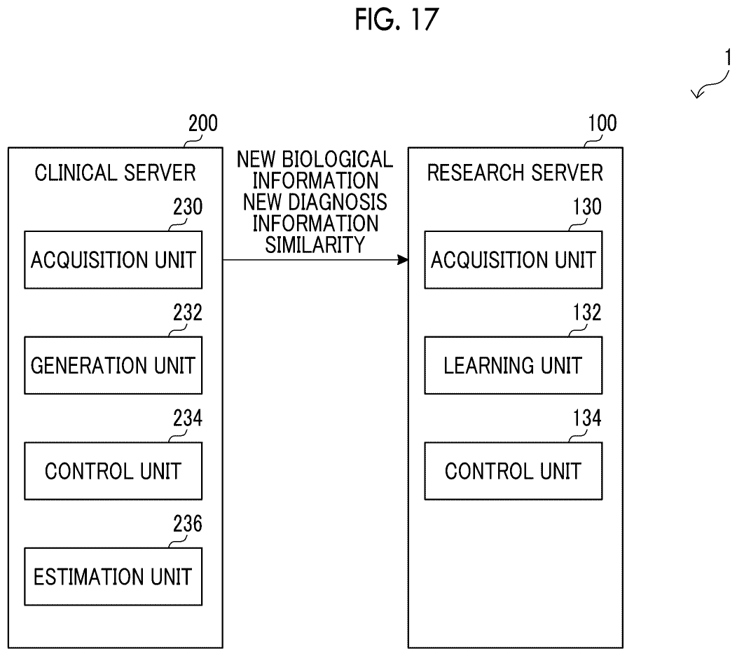

FIG. 17 illustrates an example of a functional configuration of the research server 100 and the clinical server 200 according to the present exemplary embodiment. As illustrated in FIG. 17, the research server 100 according to the present exemplary embodiment includes an acquisition unit 130, a learning unit 132, and a control unit 134, and has the same configuration as that of the first exemplary embodiment. The clinical server 200 according to the present exemplary embodiment further includes an estimation unit 236 in addition to the acquisition unit 230, the generation unit 232, and the control unit 234, as in the first exemplary embodiment. In a case where the CPU 221 executes the information processing program 227, the CPU 221 functions as the acquisition unit 230, the generation unit 232, the control unit 234, and the estimation unit 236.

### (Operation Phase)

The estimation unit 236 of the clinical server 200 estimates a similarity between the accessory information attached to the biological information (training data) used for the training of the diagnosis model M and the accessory information attached to the new biological information (hereinafter, simply referred to as a “similarity”). The accessory information is stored in the biological information DB 228 together with, for example, the biological information and the new biological information.

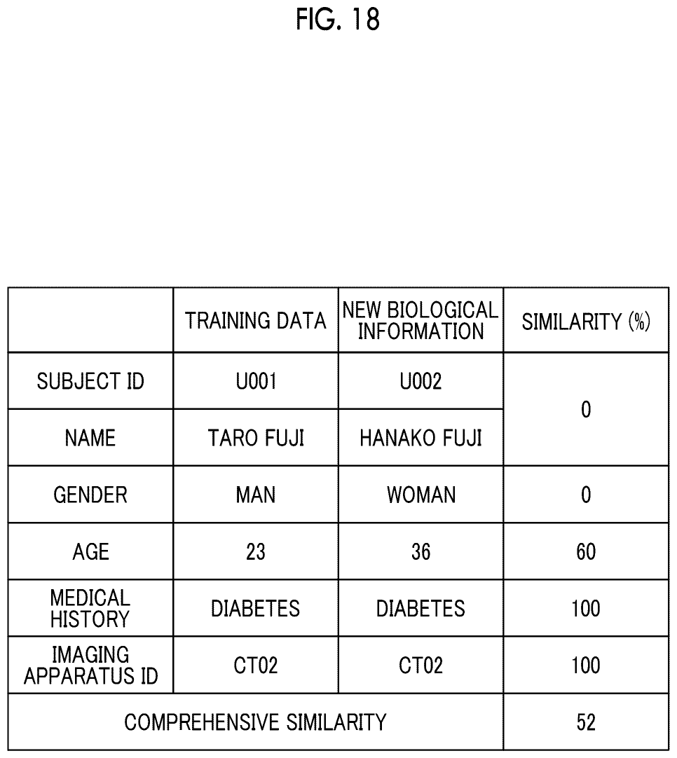

FIG. 18 illustrates an example of the accessory information of the training data and the new biological information. As illustrated in FIG. 18, the estimation unit 236 may estimate whether or not the contents are similar for each item of the accessory information, and may estimate an average of the similarities as a comprehensive similarity. In addition, for example, the estimation unit 236 may estimate a weighted average of the similarities for each item of the accessory information as a comprehensive similarity.

Note that, although only one piece of training data is illustrated in FIG. 18, in reality, a plurality of pieces of training data exist. Therefore, the estimation unit 236 may estimate a similarity between the accessory information of the new biological information and the accessory information of each of the plurality of pieces of training data, and may estimate a representative value such as an average value or a median value of the similarities, as a comprehensive similarity.

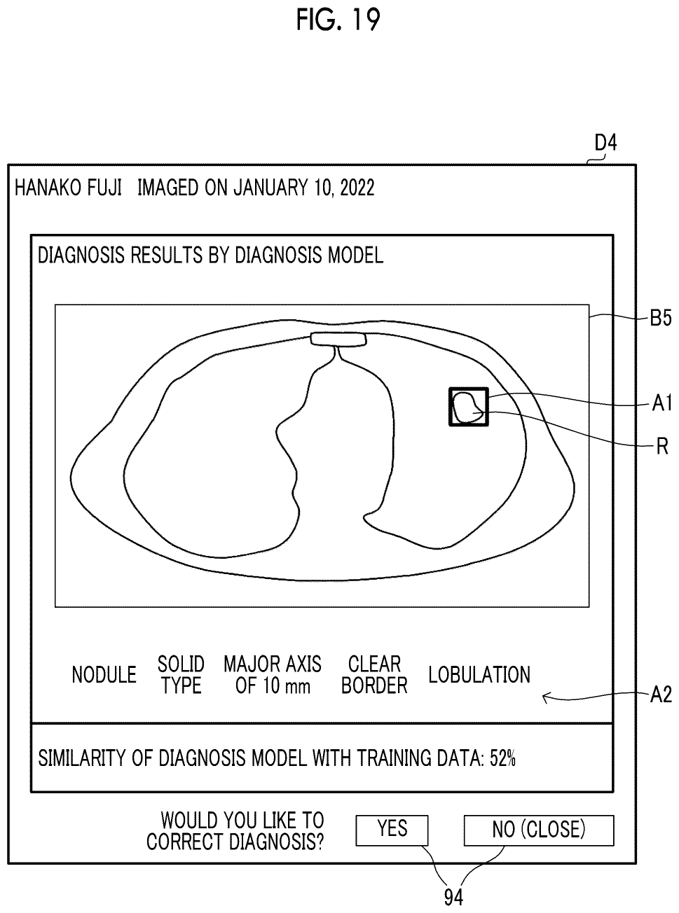

The control unit 234 of the clinical server 200 may present the new diagnosis information generated by the generation unit 232 and the similarity estimated by the estimation unit 236. FIG. 19 illustrates an example of a screen D4 displayed on the display 224 by the control unit 234. On the screen D4, the similarity estimated by the estimation unit 236 is displayed in addition to the new diagnosis information as in the screen D2 (refer to FIG. 8). It is considered that, as the similarity is higher, the new biological information of the diagnosis target is more suitable for the diagnosis model M and the accuracy of the new diagnosis information is higher. Therefore, by presenting the similarity, the user can understand the accuracy of the new diagnosis information. In addition, the control unit 234 stores the new biological information, the new diagnosis information, and the similarity in the biological information DB 228 of the storage unit 222 in association with each other.

### (Retraining Phase)

In addition, in the retraining phase, the control unit 234 of the clinical server 200 outputs the new diagnosis information, the new biological information, and the similarity stored in the biological information DB 228 to the research server 100. The acquisition unit 130 of the research server 100 acquires a combination of the new biological information, the new diagnosis information, and the similarity. The learning unit 132 of the research server 100 may determine whether or not to perform retraining of the diagnosis model M by using the combination of the new biological information and the new diagnosis information based on the similarity acquired by the acquisition unit 130.

Specifically, the learning unit 132 may determine whether or not to perform retraining of the diagnosis model M using the combination of the new biological information and the new diagnosis information, by comparing the similarity with a predetermined threshold value. For example, in a case where the similarity is equal to or higher than the threshold value, it can be estimated that the combination of the new biological information and the new diagnosis information is likely to be similar to the training data (the combination of the biological information and the diagnosis information) already used for the training of the diagnosis model M. On the other hand, in a case where the similarity is lower than the threshold value, it can be estimated that the combination of the new biological information and the new diagnosis information is not likely to be similar to the training data already used for the training of the diagnosis model M. In that respect, the learning unit 132 may set different conditions for whether or not the combination is used for the retraining in a case where the similarity is equal to or higher than the threshold value or whether or not the combination is used for the retraining in a case where the similarity is lower than the threshold value, according to the characteristic of the diagnosis model M that is to be trained.

For example, as in a case of creating a model specialized in a specific condition, it may be preferable to preferentially use the training data that matches a specific condition. In this case, the accuracy can be improved by performing the retraining of the diagnosis model M by using the combination of the new biological information and the new diagnosis information in which the similarity is equal to or higher than the threshold value. On the other hand, in this case, the combination of the new biological information and the new diagnosis information in which the similarity is lower than the threshold value may be noise. As a result, in a case where the retraining of the diagnosis model M is performed using the combination, the accuracy may be decreased. In a case where the similarity acquired by the acquisition unit 130 is equal to or higher than a predetermined threshold value, the learning unit 132 may perform the retraining of the diagnosis model M using the combination of the new biological information and the new diagnosis information. That is, in a case where the similarity acquired by the acquisition unit 130 is lower than the predetermined threshold value, the learning unit 132 may not use the combination of the new biological information and the new diagnosis information in the retraining of the diagnosis model M.

In addition, for example, in a case of creating a general-purpose model that can correspond to various patterns, it may be preferable to use the training data including various variations. In this case, by performing the retraining of the diagnosis model M using the combination of the new biological information and the new diagnosis information in which the similarity is lower than the threshold value, it is possible to create a model having higher generality. On the other hand, in this case, in a case where the retraining of the diagnosis model M is performed by using the combination of the new biological information and the new diagnosis information in which the similarity is equal to or higher than the threshold value, the generality of the model may be lowered. Thus, in a case where the similarity acquired by the acquisition unit 130 is lower than the predetermined threshold value, the learning unit 132 may perform retraining of the diagnosis model M by using the combination of the new biological information and the new diagnosis information. That is, in a case where the similarity acquired by the acquisition unit 130 is equal to or higher than the predetermined threshold value, the learning unit 132 may not use the combination of the new biological information and the new diagnosis information in the retraining of the diagnosis model M.

The condition used by the learning unit 132 (whether or not the combination is used for the retraining in a case where the similarity is equal to or higher than the threshold value, or whether or not the combination is used for the retraining in a case where the similarity is lower than the threshold value) may be predetermined by, for example, being associated with the diagnosis model M, or may be optionally selected by the user. In addition, for example, the learning unit 132 may perform the retraining of the diagnosis model M by using the combination of the new biological information and the new diagnosis information with a weight according to the similarity.

### Third Exemplary Embodiment

The information processing system 1 according to the present exemplary embodiment has a function of anonymizing at least a part of the accessory information attached to the biological information which is used for training of the diagnosis model M and the accessory information attached to the new biological information which is used for retraining. As illustrated in FIG. 5, the accessory information may include personal information such as a name, a gender, and an age of the subject, and it is preferable not to output the personal information that does not affect the diagnosis to the research server 100 from the viewpoint of privacy protection. In the following description, the information processing system 1 according to the third exemplary embodiment will be described. On the other hand, descriptions of the same configurations and functions as those of the first and second exemplary embodiments will be omitted as appropriate.

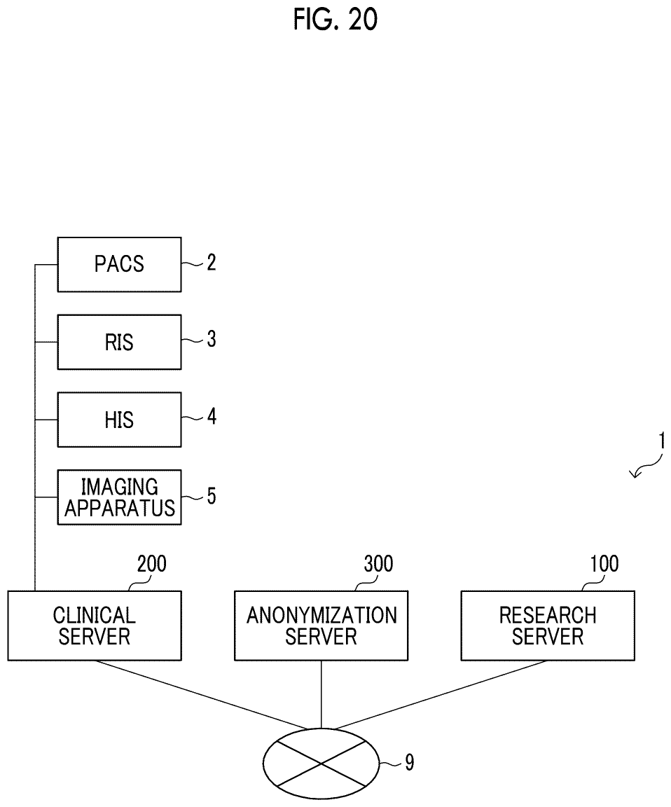

FIG. 20 is a diagram illustrating a schematic configuration of the information processing system 1 according to the present exemplary embodiment. As illustrated in FIG. 20, the information processing system 1 according to the present exemplary embodiment further includes an anonymization server 300 in addition to the information processing system 1 according to the first exemplary embodiment (refer to FIG. 1). The research server 100, the clinical server 200, and the anonymization server 300 are connected to each other via a wired or wireless network 9 such that communication can be performed. The anonymization server 300 is an example of a third information processing apparatus according to the present disclosure.

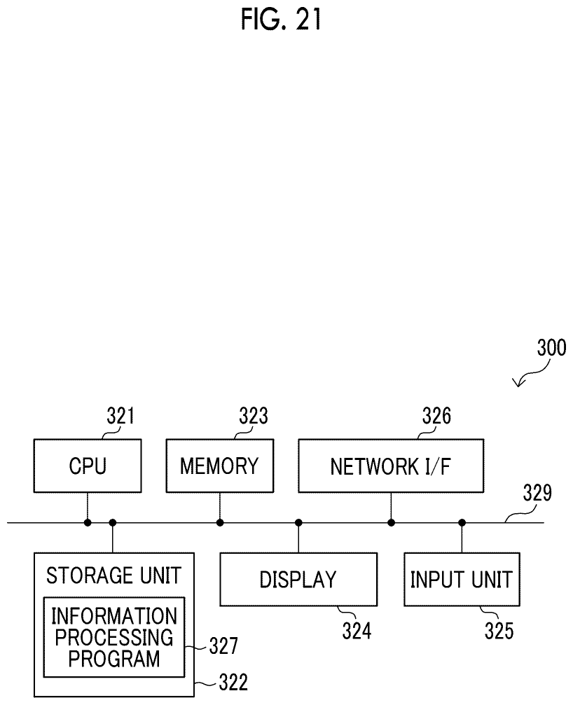

An example of a hardware configuration of the anonymization server 300 will be described with reference to FIG. 21. As the anonymization server 300, for example, a server computer, a personal computer, or the like can be appropriately applied. As illustrated in FIG. 21, the anonymization server 300 includes a CPU 321, a non-volatile storage unit 322, and a memory 323 as a temporary storage region. In addition, the anonymization server 300 includes a display 324 such as a liquid crystal display, an input unit 325 such as a keyboard and a mouse, and a network I/F 326. The network I/F 326 is connected to the network 9, and performs wired or wireless communication. The CPU 321, the storage unit 322, the memory 323, the display 324, the input unit 325, and the network I/F 326 are connected to each other via a bus 329 such as a system bus and a control bus such that various types of information can be exchanged.

The storage unit 322 is realized by, for example, a storage medium, such as an HDD, an SSD, and a flash memory. The storage unit 322 stores an information processing program 327 in the anonymization server 300. The CPU 321 reads out the information processing program 327 from the storage unit 322, develops the read information processing program 327 in the memory 323, and executes the developed information processing program 327. The CPU 321 is an example of a third processor according to the present disclosure.

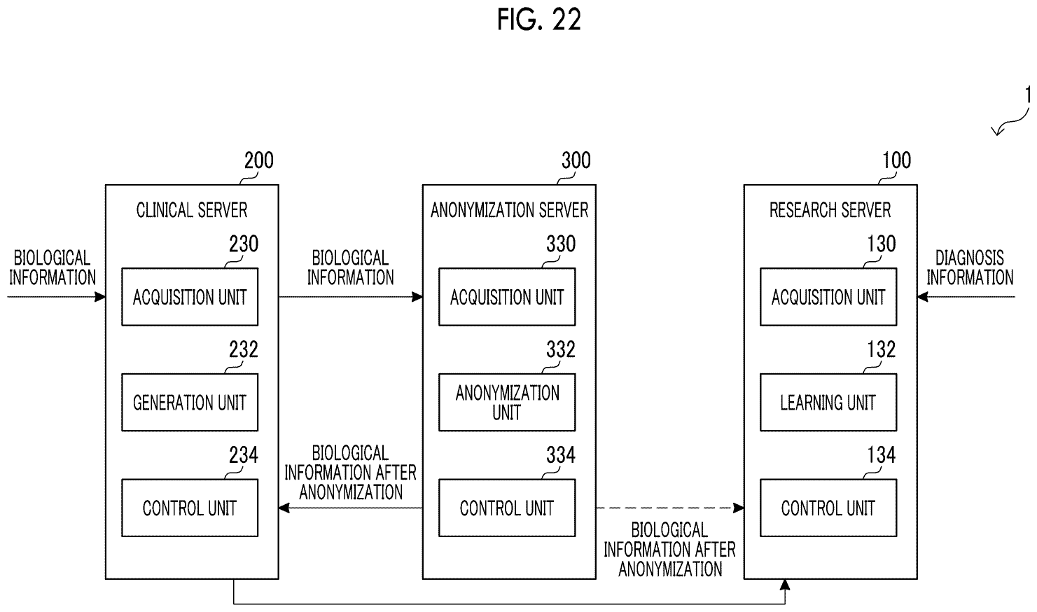

An example of a functional configuration of the research server 100, the clinical server 200, and the anonymization server 300 according to the present exemplary embodiment will be described with reference to FIG. 22. As illustrated in FIG. 22, the research server 100 according to the present exemplary embodiment includes an acquisition unit 130, a learning unit 132, and a control unit 134, and has the same configuration as that of the first exemplary embodiment. The clinical server 200 according to the present exemplary embodiment includes an acquisition unit 230, a generation unit 232, and a control unit 234, and has the same configuration as that of the first exemplary embodiment.

The anonymization server 300 includes an acquisition unit 330, an anonymization unit 332, and a control unit 334. In a case where the CPU 321 executes the information processing program 327, the CPU 321 functions as the acquisition unit 330, the anonymization unit 332, and the control unit 334. FIG. 22 also illustrates various types of information to be input and output to and from the research server 100, the clinical server 200, and the anonymization server 300 in the training phase.

### (Training Phase)

First, a form of anonymizing the accessory information attached to the biological information used for the training of the diagnosis model M will be described. The acquisition unit 230 of the clinical server 200 acquires the biological information from various external apparatuses such as the PACS 2, the RIS 3, the HIS 4, and the imaging apparatus 5. Here, the accessory information including personal information such as a name, a gender, and an age of the subject is attached to the biological information. The control unit 234 of the clinical server 200 stores the biological information acquired by the acquisition unit 230 in the biological information DB 228 of the storage unit 222, together with the accessory information. In addition, the control unit 234 outputs the biological information stored in the biological information DB 228 to the anonymization server 300 together with the accessory information.

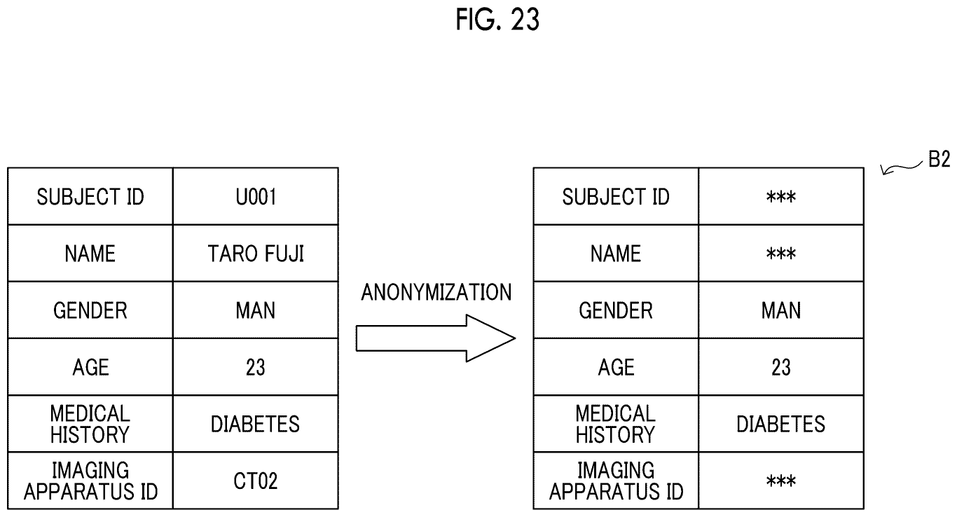

The acquisition unit 330 of the anonymization server 300 acquires the biological information and the accessory information from the clinical server 200. The anonymization unit 332 of the anonymization server 300 anonymizes at least a part of the accessory information attached to the biological information acquired by the acquisition unit 330. FIG. 23 illustrates an example of the accessory information in which a part is anonymized. As illustrated in FIG. 23, the anonymization unit 332 may hide the personal information that does not affect the diagnosis, such as a subject ID and a name. On the other hand, the anonymization unit 332 may not anonymize information that can be used for diagnosis, such as gender, age, and medical history, even though the information is personal information. An item to be anonymized in the accessory information may be, for example, predetermined, or may be optionally designated via the input unit 325.

The control unit 334 of the anonymization server 300 outputs the biological information to which the accessory information anonymized by the anonymization unit 332 is attached to the clinical server 200. The acquisition unit 230 of the clinical server 200 acquires the biological information to which the accessory information after anonymization is attached from the anonymization server 300. The control unit 234 of the clinical server 200 stores the biological information acquired by the acquisition unit 230, the accessory information before anonymization, and the accessory information after anonymization in the biological information DB 228 of the storage unit 222 in association with each other.

The acquisition unit 130 of the research server 100 acquires the biological information to which the accessory information after anonymization is attached from the clinical server 200. Thereafter, the research server 100 receives the diagnosis information and performs training of the diagnosis model M in the same manner as in the first exemplary embodiment.

### (Retraining Phase)

Next, a form of anonymizing the accessory information attached to the new biological information to be used for the retraining of the diagnosis model M will be described. The acquisition unit 330 of the anonymization server 300 acquires the accessory information attached to the new biological information from the clinical server 200. The anonymization unit 332 of the anonymization server 300 anonymizes at least a part of the accessory information attached to the new biological information acquired by the acquisition unit 330.

The control unit 334 of the anonymization server 300 outputs the new biological information to which the accessory information anonymized by the anonymization unit 332 is attached to the clinical server 200. The acquisition unit 230 of the clinical server 200 acquires the new biological information to which the accessory information after anonymization is attached from the anonymization server 300. The control unit 234 of the clinical server 200 stores the new biological information acquired by the acquisition unit 230, the accessory information before anonymization, the accessory information after anonymization, and the new diagnosis information in the biological information DB 228 of the storage unit 222 in association with each other.

The acquisition unit 130 of the research server 100 acquires the new biological information to which the accessory information after anonymization is attached from the clinical server 200. Thereafter, the learning unit 132 of the research server 100 performs retraining of the diagnosis model M by using the combination of the new biological information and the new diagnosis information, in the same manner as in the first exemplary embodiment.

As described above, with the information processing system 1 according to the present exemplary embodiment, in the accessory information acquired by the research server 100, the personal information is anonymized. Therefore, the training and the operation of the learning model can be consistently managed while protecting privacy.

Note that, in the third exemplary embodiment, the form in which the research server 100 acquires the biological information and the new biological information to which the accessory information after anonymization is attached from the clinical server 200 is described. On the other hand, the present disclosure is not limited thereto. For example, as illustrated by a dotted line in FIG. 22, the research server 100 may directly acquire the biological information and the new biological information to which the accessory information after anonymization is attached from the anonymization server 300.

In addition, in each of the above-described exemplary embodiments, the diagnosis model M in which the input is the biological information including the medical image and the output is the diagnosis information has been described. On the other hand, the learning model to which the technology of the present disclosure can be applied is not limited thereto. For example, the technology of the present disclosure may be applied to a learning model that performs so-called multimodal training of outputting diagnosis information based on other information related to the subject in addition to the medical image.

Specifically, the biological information, which is the input of the learning model, may include pre-diagnosis information indicating information obtained by performing diagnosis in advance in relation to the medical image included in the biological information. The pre-diagnosis information may be, for example, information described in an electronic medical record of a subject from which the medical image is acquired, a past examination result, information generated by a known CAD system, or the like. Specific examples of such information include information indicating a position and a feature amount (for example, a volume and a major axis) of the region-of-interest R included in the medical image, information indicating a grade of the disease (for example, a grade of the cancer), and a processing result of texture analysis on the medical image. In this case, in the training phase, the learning unit 132 of the research server 100 may perform training of a learning model that receives the biological information and outputs the diagnosis information by using a combination of the medical image included in the biological information and the pre-diagnosis information and the diagnosis information related to the biological information.

In addition, in each of the above-described exemplary embodiments, for example, as a hardware structure of the processing unit that executes various types of processing, such as the acquisition units 130, 230, and 330, the learning unit 132, the generation unit 232, the anonymization unit 332, the control units 134, 234, and 334, and the estimation unit 236, various processors can be used as follows. As described above, in addition to the CPU that is a general-purpose processor that executes software (program) to function as various processing units, the various processors include a programmable logic device (PLD) that is a processor of which a circuit configuration can be changed after manufacture, such as a field programmable gate array (FPGA), and a dedicated electric circuit that is a processor having a circuit configuration that is designed for exclusive use in order to execute a specific process, such as an application specific integrated circuit (ASIC).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which the plurality of processing units are configured by one processor, firstly, as represented by a computer such as a client and a server, a form in which one processor is configured by a combination of one or more CPUs and software and the processor functions as the plurality of processing units may be adopted. Second, as represented by a system on chip (SoC) or the like, there is a form in which the processor is used in which the functions of the entire system which includes the plurality of processing units are realized by a single integrated circuit (IC) chip. In this manner, the various processing units are configured by using one or more various processors as a hardware structure.

Further, the hardware structure of these various processors is, more specifically, an electric circuit (circuitry) in which circuit elements such as semiconductor elements are combined.

Further, in each of the above exemplary embodiments, the form in which the information processing programs 127, 227, and 327 are respectively stored (installed) in the storage units 122, 222, and 322 in advance has been described. On the other hand, the present disclosure is not limited thereto. The information processing programs 127, 227, and 327 may be provided by being recorded in a recording medium such as a compact disc read only memory (CD-ROM), a digital versatile disc read only memory (DVD-ROM), or a Universal Serial Bus (USB) memory. Further, the information processing programs 127, 227, and 327 may be downloaded from an external apparatus via a network. Furthermore, in addition to the information processing program, the technology of the present disclosure is applied to a storage medium that stores the information processing program in a non-transitory manner.

The technology of the present disclosure can also appropriately combine the above-described exemplary embodiments. The content of the above description and the content of the drawings are detailed description of portions according to the technology of the present disclosure, and are merely examples of the technology of the present disclosure. For example, the above description related to the configuration, the function, the action, and the effect is the description related to the examples of the configuration, the function, the action, and the effect of the parts according to the technology of the present disclosure. Therefore, it goes without saying that, in the described contents and illustrated contents, unnecessary parts may be deleted, new components may be added, or replacements may be made without departing from the spirit of the technology of the present disclosure.

In the disclosure of Japanese Patent Application No. 2022-019036, filed Feb. 9, 2022, the entire contents of which are incorporated herein by reference. All documents, Patent Applications, and technical standards described in this specification are incorporated herein by reference to the same extent as in a case where each document, each Patent Application, and each technical standard are specifically and individually described by being incorporated by reference.

What is claimed is:

1. An information processing system comprising:
a first information processing apparatus including at least one first processor; and
a second information processing apparatus including at least one second processor, wherein:
the first processor is configured to perform training of a learning model that receives biological information and outputs diagnosis information by using a combination of biological information including at least a medical image and diagnosis information related to the biological information, and
the second processor is configured to:
acquire the learning model that is trained from the first information processing apparatus; and
generate new diagnosis information related to new biological information that is different from the biological information used for training of the learning model that is trained by inputting the new biological information to the learning model that is trained.

2. The information processing system according to claim 1, wherein:
the first information processing apparatus further includes an input unit,
the second information processing apparatus further includes a storage unit that stores the biological information, and
the first processor is configured to:
acquire the biological information stored in the storage unit from the second information processing apparatus;
display the biological information on a display; and
receive, via the input unit, an input for diagnosis information related to the displayed biological information.

3. The information processing system according to claim 1, wherein the first processor is configured to:
acquire a combination of the new biological information and the new diagnosis information; and
perform retraining of the learning model by using the combination of the new biological information and the new diagnosis information.

4. The information processing system according to claim 1, wherein:
the second information processing apparatus further includes an input unit, and the second processor is configured to:
display the generated new diagnosis information on a display; and
receive, via the input unit, a correction for the new diagnosis information.

5. The information processing system according to claim 4, wherein the first processor is configured to:
acquire a combination of the new biological information and the new diagnosis information; and
perform, in a case where the new diagnosis information is corrected, retraining of the learning model by using a combination of the new biological information and the corrected new diagnosis information.

6. The information processing system according to claim 1, wherein accessory information is attached to the biological information, the accessory information indicating information related to at least one of a subject from which the biological information is acquired or an imaging apparatus used for acquisition of the biological information.

7. The information processing system according to claim 6, further comprising a third information processing apparatus including at least one third processor, wherein
the third processor is configured to anonymize at least a part of the accessory information attached to the biological information.

8. The information processing system according to claim 7, wherein:
the second information processing apparatus further includes a storage unit that stores the biological information and the accessory information,
the third processor is configured to:
acquire the accessory information from the second information processing apparatus; and
anonymize at least a part of the accessory information,
the second processor is configured to:
acquire the accessory information after anonymization from the third information processing apparatus; and
store, in the storage unit, the biological information, the accessory information before anonymization, and the accessory information after anonymization in association with each other, and
the first processor is configured to acquire the biological information to which the accessory information after anonymization is attached.

9. The information processing system according to claim 7, wherein:
the third processor is configured to:
acquire accessory information attached to the new biological information from the second information processing apparatus; and
anonymize at least a part of the accessory information, and
the first processor is configured to:
acquire the new biological information to which the accessory information after anonymization is attached; and
perform retraining of the learning model by using a combination of the new biological information and the new diagnosis information.

10. The information processing system according to claim 6, wherein the second processor is configured to estimate a similarity between the accessory information attached to the biological information used for training of the learning model and the accessory information attached to the new biological information.

11. The information processing system according to claim 10, wherein the second processor is configured to present the generated new diagnosis information and the estimated similarity.

12. The information processing system according to claim 10, wherein the first processor is configured to:
acquire a combination of the new biological information, the new diagnosis information, and the similarity; and
determine whether or not to perform retraining of the learning model by using the combination of the new biological information and the new diagnosis information based on the similarity.

13. The information processing system according to claim 6, wherein the accessory information includes information indicating at least one of a name, a gender, an age, a medical history, and an identification number of the subject, or an imaging condition used for acquiring the biological information.

14. The information processing system according to claim 1, wherein:
the biological information includes pre-diagnosis information indicating information obtained by performing diagnosis in advance in relation to the medical image included in the biological information, and
the first processor is configured to perform training of the learning model that receives the biological information and outputs the diagnosis information by using a combination of the medical image included in the biological information, the pre-diagnosis information, and the diagnosis information related to the biological information.

15. The information processing system according to claim 1, wherein the diagnosis information includes at least one of information indicating a position and a size of a region-of-interest included in the medical image or information indicating an opinion of the region-of-interest.

16. An information processing method comprising:
causing a first processor to perform training of a learning model that receives biological information and outputs diagnosis information by using a combination of biological information including at least a medical image and diagnosis information related to the biological information; and
causing a second processor to
acquire the learning model that is trained by the first processor, and
generate new diagnosis information related to new biological information, which is different from the biological information used for training of the learning model that is trained, by inputting the new biological information to the learning model that is trained.

17. A non-transitory computer-readable storage medium storing an information processing program causing:
a first processor to execute processing of performing training of a learning model that receives biological information and outputs diagnosis information by using a combination of biological information including at least a medical image and diagnosis information related to the biological information; and
a second processor to execute processing of
acquiring the learning model that is trained by the first processor, and
generating new diagnosis information related to new biological information, which is different from the biological information used for training of the learning model that is trained, by inputting the new biological information to the learning model that is trained.
